(19) United States
(12) Patent Application Publication
(10) Pub. No.: US 2026/0249603 A1
(43) Pub. Date: Aug. 27, 2026

(54) SYSTEM AND METHOD FOR FORMING A FABRIC SHEET-LIKE PANEL HAVING A WETNESS INDICATOR

(71) Applicant: Henkel AG & Co. KGaA, Düsseldorf (DE)

(72) Inventors: Haie Deng, Guangdong (CN); Weiyi Li, Guangdong (CN)

(21) Appl. No.: 19/652,027

(22) Filed: Apr. 20, 2026

Related U.S. Application Data
(63) Continuation of application No. PCT/CN2023/128144, filed on Oct. 31, 2023.

(51) Int. Cl. B32B 37/12; B32B 5/02; B32B 37/00; B32B 37/06; B32B 37/16; B32B 38/18

(52) U.S. Cl. CPC B32B 37/1292; B32B 5/02; B32B 37/0053; B32B 37/06; B32B 37/16; B32B 38/18; B32B 2037/1215; B32B 2250/02; B32B 2305/18; B32B 2309/02

(57) ABSTRACT

The present application generally discloses a system and a method for forming a fabric sheet-like panel having a wetness indicator. The system comprises a transfer unit (100) configured to receive a functional film (2000); and a laminating unit (200) arranged downstream of the transfer unit (100) and configured to receive a fabric substate (1000) and laminate the same together with the functional film (2000) as the fabric sheet-like panel. The transfer unit (100) includes a rotatable transfer roller (121) and a fluid holding tank (122) fixedly disposed adjacent to the transfer roller (121), the fluid holding tank (122) is configured to store a wetness indicator which can be also as a hot melt adhesive, such that as the transfer roller (121) rotates, the wetness indicator is able to be applied in a patten including irregular curves onto a side of the functional film (2000) facing the fabric substrate (1000). During application of the wetness indicator, the transfer roller (121) and/or the fluid holding tank (122) can be selectively heated such that a temperature of the transfer roller (121) is kept between 80° C. and 100° C. and a temperature of the fluid holding tank (122) is kept greater than 80° C.

### FIELD

The present application generally relates to a system and method for forming a fabric sheet-like panel having a wetness indicator.

### BACKGROUND

In medical fabric sheet-like panels, a wetness indicator may be added in a specific-pattern. Such a wetness indicator is a fluidic agent which can be fluidically applied onto a fabric sheet-like panel. When the fluidic agent is cured, it is transparent. Therefore, the medical fabric sheet-like panel can be laid as a bed sheet onto a bed of a patient. When the bed is dry, the medical fabric sheet-like panel has no difference than an ordinary bed sheet. When the wetness indicator is wet by water, urine or other liquids, the wetness indicator can be changed from its transparent state to an opaque state in which the wetness indicator can be colored or darkened, to visually alter nurses that there exists an abnormal condition on the bed. Usually, the wetness indicator is fluidically applied in several straight lines only.

Like the medical fabric sheet-like panels, diapers or pants-type diapers are also usually formed by several fabric sheet-like panels sewn or bonded together in which a wetness indicator is added. However, different than medical uses of the medical fabric sheet-like panels, most diapers or pants-type diapers are mainly prepared for child-clients. In this case, a pattern comprised of straight lines only may not be sufficient to visually attract parents to notice the wetness indicator in its opaque state. Therefore, it is hoped that the pattern of the wetness indicator can become more complex such that when it is wet, it is more visually attractive for parents to know than it is time to replace the wet diapers or pants-type diapers with new ones. This is because in a fixed area a complicated pattern of the wetness indicator in the opaque state will look more conspicuous than a simple pattern (like the one of straight lines only). Of course, the wetness indicator can be configured such that it is colored when it is wet; and it is colorless when it is dry.

The complicated pattern may include sinuous lines or irregular curves, or for example may be in the form of a cute animal, a flower, a cartoon character or any other interested figure. In this case, a traditional system of method for applying the wetness indicator will not be suitable for the complicated pattern. This is because the sinuous lines or irregular curves will result in that it is difficult to apply the wetness indicator evenly, smoothly, and readily by a transfer roller. Besides, the wetness indicator can be used as a hot melt adhesive to bond a functional film onto a fabric sheet-like panel. If the pattern of the wetness indicator is too complicated, undesired wrinkles will occur due to unsuitable temperature control of the applied wetness indicator. The wrinkles will significantly affect the quality of the finished fabric sheet-like panel.

### SUMMARY

To solve the above technical issues, the present application is aimed at proposing an improved system and method for applying a wetness indicator to form a sheet-like panel, especially a fabric sheet-like panel, in which the wetness indicator is applied evenly, smoothly, and readily in a relatively complicated pattern (not comprised of straight lines only).

According to one aspect, the present application proposes a system for forming a fabric sheet-like panel having a wetness indicator, comprising:
a transfer unit configured to receive a functional film; and
a laminating unit arranged downstream of the transfer unit and configured to receive a fabric substate and laminate the same together with the functional film as the fabric sheet-like panel, wherein
the transfer unit includes a rotatable transfer roller and a fluid holding tank fixedly disposed adjacent to the transfer roller, the fluid holding tank is configured to store a wetness indicator which can be also as a hot melt adhesive, such that as the transfer roller rotates, the wetness indicator is able to be applied in a patten including irregular curves onto a side of the functional film facing the fabric substrate, and wherein
during application of the wetness indicator, the transfer roller and/or the fluid holding tank can be selectively heated such that a temperature of the transfer roller is kept between 80° C. and 100° C. and a temperature of the fluid holding tank is kept greater than 80° C.

In an embodiment, the wetness indicator to be applied in the transfer unit has a viscosity between 200 CPS and 2000 CPS at 90° C.

In an embodiment the transfer unit is configured such that the wetness indicator is applied at a unit weight of 5 to 25 GSM onto the side of the functional film.

In an embodiment, the temperature of the transfer roller is kept between 80° C. and 90° C. and the temperature of the fluid holding tank is kept between 80° C. and 130° C.

In an embodiment, the fabric substrate is a non-woven fabric substrate and the functional film is a PE film.

In an embodiment, the transfer unit also comprise a drive roller which can rotate in a direction opposite to the transfer roller, the PE film received by the transfer unit is clamped and moved between the drive roller and the transfer roller, and the transfer roller is on its outer surface formed with a pattern mirror relative to the pattern of the wetness indicator to be applied, and using the mirrored pattern, the wetness indicator is transferred onto the PE film.

In an embodiment, the pattern of the wetness indicator is shaped as a figure which includes a sinuous line or an irregular line and is interested by children. For example, the figure includes but is not limited to a cute animal like a cute lion, a flower, a cartoon character, or any other interesting figure.

In an embodiment, the mirrored pattern is embossed or recessed on the outer surface of the transfer roller.

In an embodiment, the laminating unit comprises a pressurizing roller and an additional drive roller which rotate in opposite directions such that both the non-woven fabric substrate and the PE film are received and laminated through between the two rollers.

According to another aspect, the present application proposes a method for forming a fabric sheet-like panel having a wetness indicator, comprising:
providing a system including a transfer unit configured to receive a functional film and a laminating unit arranged downstream of the transfer unit and configured to receive a fabric substate and laminate the same together with the functional film;
providing a wetness indicator which has a viscosity between 200 CPS and 2000 CPS at 90° C. and storing it in a fluid holding tank of the transfer unit;
moving the functional film through the transfer unit such that the wetness indicator can be applied by a transfer roller of the transfer unit and the fluid holding tank, in a patten including irregular curves, onto a side of the functional film which will face the fabric substrate in the laminating unit; and
during application of the wetness indicator, keeping a temperature of the transfer roller between 80° C. and 100° C. and keeping a temperature of the fluid holding tank (122) greater than 80° C.

In an embodiment, in the transfer module the wetness indicator is applied at a unit weight of 5 to 25 GSM onto the side of the functional film.

In an embodiment, the temperature of the transfer roller is kept between 80° C. and 90° C. and the temperature of the fluid holding tank is kept between 80° C. and 130° C.

In an embodiment, the fabric substrate is a non-woven fabric substrate and the functional film is a PE film.

In an embodiment, the pattern of the wetness indicator is shaped as a figure which includes a sinuous line or an irregular line and is interested by children. For example, the figure of the pattern includes but is not limited to a cute animal like a cute lion, rabbit, a flower, or a cartoon character or any other interesting figure.

Using the above technical measures, the wetness indicator can be provided in a relatively complicated pattern, for example a unique and attractive pattern, within sheet-like panels used to form diapers or pants-type diapers. The system and method according to the present application will enable the wetness indicator in its fluidic state to be transferred smoothly and easily by the transfer roller to support the complicated pattern to be applied. Besides, according to the present application, temperature control during application of the wetness indicator will not allow wrinkle existing on the functional film to be laminated with the non-woven fabric panel, such that the quality of the final fabric sheet-like panel can be enhanced.

### BRIEF DESCRIPTION OF THE DRAWINGS

Various aspects and embodiments of the present application disclosed herein are described below with reference to the accompanying figures. It should be appreciated that the figures are not necessarily drawn to scale. Items appearing in multiple figures may be indicated by the same reference numeral. For the purposes of clarity, not every component may be labeled in every figure.

FIG. 1 is a block diagram schematically illustrating a system for forming a fabric sheet-like panel provided with a wetness indicator according to an embodiment of the present application;

FIG. 2 is a perspective view schematically illustrating a part of the system according to the embodiment of the present application;

FIG. 3A is a diagram schematically illustrating a pattern of the wetness indicator in the fabric sheet-like panel, which wetness indicator is in a dry state;

FIG. 3B is a diagram schematically illustrating the pattern of the wetness indicator in the fabric sheet-like panel, which wetness indicator is in a wetted state; and

FIG. 4 is a flow chart schematically illustrating a method for forming the fabric sheet-like panel provided with the wetness indicator according to an embodiment of the present application.

### EMBODIMENTS

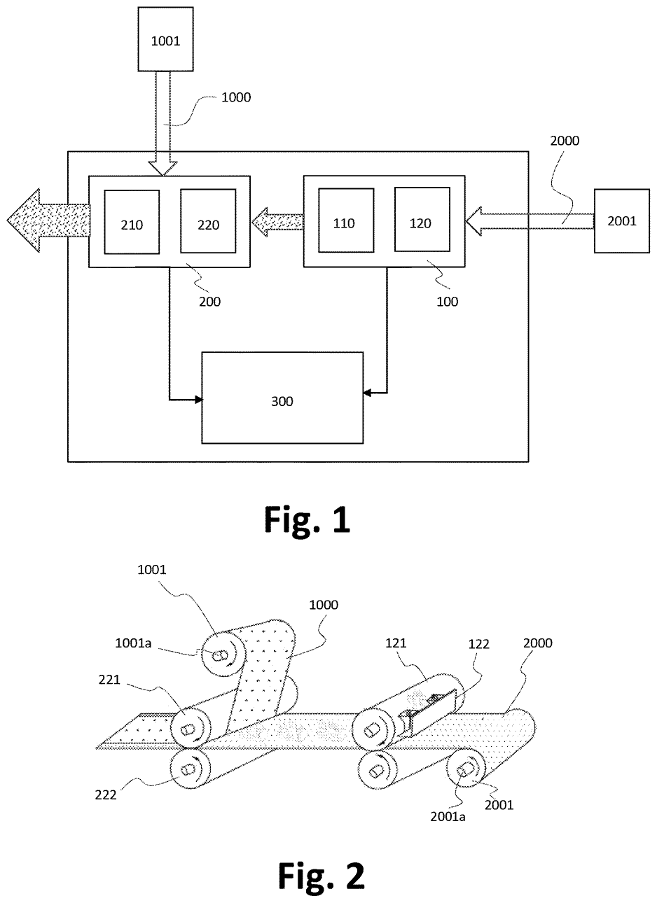

FIG. 1 schematically illustrates a system according to an embodiment of the present application configured to receive a first fabric substate 1000 from a fabric substrate source 1001 and a second functional film 2000 from a film source 2001 and then laminate them together to form a fabric sheet-like panel. The fabric sheet-like panel is provided with a wetness indicator and can be used to produce diapers or pants-type diapers. In the embodiment, the fabric substrate source 1001 can be a non-woven fabric substrate roll and thus the first fabric substrate 1000 can be a non-woven fabric substrate; and the film source 2001 can be a polyethylene (PE) film roll and thus the functional film 2000 can be a PE film.

According to the embodiment, the system generally comprises a transfer unit 100 and a laminating unit 200 arranged downstream of the transfer unit 100. The transfer unit 100 is configured to apply the wetness indicator, which is in a fluidic mode, onto the PE film. In the context of the present application, the wetness indicator can be a pressure sensitive adhesive or hot melt adhesive having a wetness indicating function. That is to say, the wetness indicator in its fluidic mode can be used to bond the non-woven fabric substrate 1000 and the PE film 2000 together and in its cured mode can be used to indicate a wetting state (by changing its transparent state to its opaque or colored state) when it is wetted.

The transfer unit 100 is provided with a drive module 110 and an applying module 120. The drive module 110 is configured to receive the PE film 2000 from the PE film roll 2001 and move the same towards the laminating unit 200. The applying module 120 is configured to apply the wetness indicator onto the moving PE film in a specific pattern, for example a relatively complicated pattern, or a pattern not comprised of straight lines only. The applying module 120 can be provided with a heater module (not shown) configured to selectively heat the wetness indicator to be applied, and a temperature sensing module (not shown) configured to detect a temperature of the wetness indicator to be applied. In the system, a central control unit 300 is provided to control a speed at which the PE film 1000 is moved through the transfer unit 100. In the meanwhile, the central control unit 300 is electrically connected to the heater module and the temperature sensing module such that the temperature of the wetness indicator to be applied can be controlled correspondingly.

The laminating unit 200 is provided with a drive module 210 and a pressurizing module 220. The drive module 210 is configured to move the PE film 2000 and the non-woven fabric substrate 1000 together towards an outlet (not shown) of the system. The pressurizing module 220 is configured to exert a pressure between the moving PE film 2000 and non-woven fabric substrate 1000 such that they can be laminated with together as a final fabric sheet-like panel. It should be conceived that before the PE film 2000 enters the laminating unit 200 the wetness indicator is applied onto a side of the PE film 2000 facing the non-woven fabric substrate 1000. The central control unit 300 is configured to control the exerted pressure and a speed at which the PE film 1000 and the non-woven fabric substrate 2000 are moved through the laminating unit 200. Therefore, the central control unit 300 can be a computer system having a memory therein. In the memory, one or more specific programs can be stored. For example, the programs can be coded depending on a method according to the present application which will be explained below, and, when necessary, can be invoked and executed by the computer system.

FIG. 2 schematically illustrates a part of one concrete example of the system according to the embodiment of the present application. It should be understood by a person skilled in the art that the example as shown is non-limited and for illustrative purpose only. The system or its units or modules according to the present application can be embodied in any other suitable form as soon as the basic principle as explained below can be achieved. As shown, the PE film roll 2001 is free-rotationally supported about a stationary shaft 2001a. In this example, the drive module 110 comprises a drive roller which is configured to rotate in the same direction as the PE film roll 2001. Moreover, the applying module 120 comprises a transfer roller 121 and a fluid holding tank 122 fixedly disposed adjacent to the transfer roller 121. The transfer roller 121 is configured to have a pattern on its outer surface which pattern is mirrored relative to a pattern of the wetness indicator to be fluidically applied onto the PE film 2000. For instance, the mirrored pattern of the transfer roller 121 can be embossed or recessed on its outer surface. The fluid holding tank 122 is configured to be filled with the wetness indicator in its fluidic mode, and has an opening facing the outer surface of the transfer roller 121. The fluid holding tank 122 is disposed such that it does not affect rotation of the transfer roller 121. Besides, the fluid holding tank 122 is equipped with a scraper (not shown) by which redundant wetness indicator on the transfer roller 121 can be returned to the fluid holding tank 122.

The PE film 2000 is configured to pass through between the transfer roller 121 and the drive roller of the drive module 110. For example, the drive roller can be a driving roller which is rotatable about its axis and the transfer roller 121 can be a driven roller which is rotatable about its axis. Boths axes of the two rollers are parallel to each other, and the PE film 2000 passing through between the two rollers is configured to press against either of them. The fluid holding tank 122 is disposed above a part of the PE film 2000 intending to pass through between the two rollers. Moreover, the two rollers in the transfer unit 100 rotate in opposition directions such that the PE film 2000 can be pulled out of the PE film roll 2001, during its free rotation, and be moved towards the laminating unit 200.

Viewed in the rotational direction of the transfer roller 121, the fluid holding tank 122 is disposed upstream of a contact area of the moving PE film 2000 with the transfer roller 121. Therefore, as the transfer roller 121 rotates, the mirrored pattern thereof will be sufficiently adhered with the fluidic wetness indicator. The adhered fluidic wetness indicator will be transferred onto the moving PE film 2000 at the contact area (as indicated by FIG. 2), especially onto an upper side of the moving PE film 2000 at the contact area, such that the desired pattern of the wetness indicator can exist on the PE film 2000. The fluid holding tank 122 is configured such that without through the mirrored patten of the transfer roller 121 the fluidic wetness indicator cannot drop onto the moving PE film 2000. According to the present application, the wetness indicator as the hot melt adhesive in the transfer unit 200 is applied at a unit weight of 5 to 25 Gramme/Square Meter (GSM).

According to the illustrated example, the non-woven fabric substrate roll 1001 is free-rotatably supported about a stationary shaft 1001a. The pressurizing module 220 comprises a pressurizing roller 221, and the drive module 210 comprises a drive roller 211. Like the two rollers of the transfer unit 100, the rollers 221 and 211 are configured such that the non-woven fabric substrate 1000 can be pulled out of the non-woven fabric substrate roll 1001 and be moved together with the PE film 2000, already applied with the pattern of the wetness indicator, through between the rollers 221 and 211. Here, the drive roller 211 can be a driving roller and the pressurizing roller 221 can be a driven roller. According to the present application, prior to passing between the two rollers 221 and 211 in the laminating unit 200, the non-woven fabric substrate 1000 is separated from the PE film 2000. The PE film 2000 moves towards a contact area thereof with the moving non-woven fabric substrate 1000 in such a way that the side of the PE film 2000 already applied with the wetness indicator faces the moving non-woven fabric substrate 1000. Therefore, after passing through the laminating unit 100, the PE film 2000 can be laminated together with the non-woven fabric substrate 1000 to form a fabric sheet-like panel which can be later used to produce diapers or pants-type diapers. During the lamination, the wetness indicator as a hot melt adhesive facilitates secure bonding between the PE film 2000 and the non-woven fabric substrate 1000.

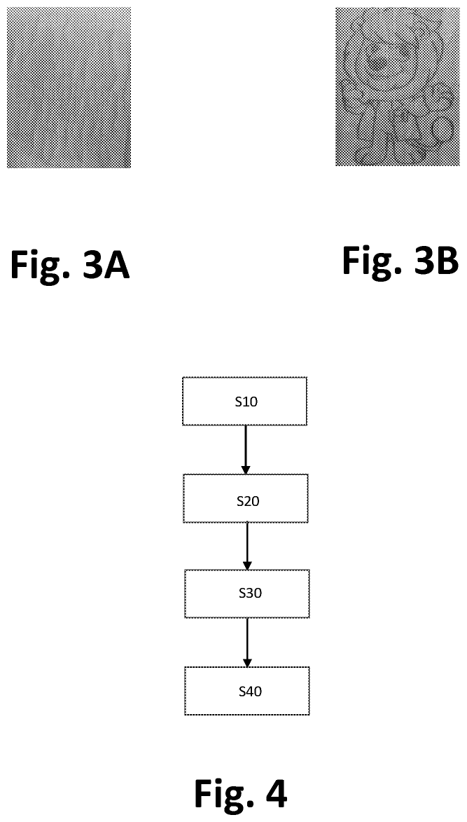

FIG. 3A schematically illustrates a pattern of the wetness indicator applied in the fabric sheet-like panel which is in a dry state and thus is transparent or almost transparent. FIG. 3B schematically illustrates the same pattern of the wetness indicator applied in the fabric sheet-like panel which is in a wetting state and is opaque or colored such that it can be visibly noticed. According to the embodiment, the pattern is shows as a cute lion, which appears to be popular with children. Of course, it can be understood by a person skilled in the art that the pattern of the wetness indicator can be embodied in any other suitable forms like rabbits, squirrels, cats, or other interesting patterns. The pattern adopted in the present application excludes straight lines only or includes straight lines and curved lines. Different than the prior art in which the pattern of the wetness indicator includes several straight lines only, the pattern of the wetness indicator according to the present application is more complicated such that a greater amount of the wetness indicator (as the hot melt adhesive) can be applied onto the PE film under the premise that the size thereof remains unchanged.

According to the present application, the transfer unit 100 is also provided with a heater module including a first heater assigned for the transfer roller 121 and a second heater assigned for the fluid holding tank 122. For instance, the first heater can be disposed within the transfer roller 121 to selectively heat the transfer roller 121 under control of the central control unit 300. For instance, again, the second heater can be disposed within the fluid holding tank 122 to selectively heat the wetness indicator (or the hot melt adhesive) within the fluid holding tank 122 under control of the central control unit 300. Moreover, the transfer unit 100 is also provided with a temperature sensing module including a first temperature sensor assigned for the transfer roller 121 and a second temperature sensor assigned for the fluid holding tank 122. The first and second temperature sensors can be used to detect the real-time temperatures of the transfer roller 121 and the fluid holding tank 122. It is conceivable that the detected real-time temperatures are directly relevant to or close to the real-time temperature of the wetness indicator (or the hot melt adhesive) to be applied at the contact area.

The inventor realizes that as the pattern of the wetness indicator to be applied becomes complicated, the viscosity and temperature of the wetness indicator to be applied might affect the quality of the final fabric sheet-like panel. Therefore, some tests are carried out in which different wetness indicators obtained in different formulas are used, the viscosities of them are varied, and the temperatures of the transfer roller 121 and the fluid holding tank 122 are monitored and controlled.

TABLE 1

| | | Temperature | Temperature | |
|---|---|---|---|---|
| | Viscosity | of the transfer | of the fluid | |
| Formula | (CPS at 90° C.) | roller | holding tank | Performance |
| 1 | 500 | 90 | 100 | ✓ |
| 1 | 500 | 100 | 100 | ✓ |
| 1 | 500 | 70 | 100 | X |
| 1 | 500 | 120 | 100 | X |
| 1 | 500 | 90 | 70 | X |
| 1 | 500 | 90 | 130 | ✓ |
| 2 | 1500 | 90 | 110 | ✓ |
| 3 | 3000 | 90 | 130 | X |
| 3 | 3000 | 110 | 130 | X |

Table 1 shows results of some tests carried out by the inventor. In the tests, the wetness indicator is produced by three different formulas. For instance, the formulas in the tests are available in the market such as Henkel DM 9860, DM 9819 and 9826U. The unit of viscosity of the wetness indicator (or the hot melt adhesive) refers to Centipoise (CPS) at 90° C. The performance refers to that whether or how the wetness indicator to be applied will affect the final fabric sheet-like sheet. This sometimes may be visually determined. For instance, if the wetness indicator can be evenly and clearly applied onto the PE film in a complicated pattern (such as the pattern as shown by FIG. 3B) and no winkle will exist on the PE film, the performance will be regarded as “V”. Otherwise, the performance will be regarded as “X”. As shown by Table 1, when the viscosity of the wetness indicator is too greater (as presented by the last two rows in Table 1), the performance will be bad. For example, when the viscosity of the wetness indicator (in formula 3) to be applied reaches 3000 CPS at 90° C. and the temperatures of the transfer roller and the tank reach 90° C. and 130° C. respectively, the applied wetness indicator through the transfer unit 100 can be observed to have a drawing phenomenon, which will negatively affect the final pattern of the applied wetness indicator in the fabric sheet-like panel. For example again, when the viscosity of the wetness indicator (in formula 3) to be applied reaches 3000 CPS at 90° C. and the temperatures of the transfer roller and the tank reach 110° C. and 130° C. respectively, the PE film is observed to be burnt. This might be caused by the higher viscosity and the higher temperature of the wetness indicator. Especially, when the temperature of the transfer roller is too high, the PE film will wrinkle due to its natural property. Of course, when the viscosity of the wetness indicator is too low, the applied wetness indicator will most likely penetrate through the moving non-woven fabric substrate 1000 in the laminating unit 200 such that the pressurizing roller 221 is contaminated by the penetrating wetness indicator.

Through all the tests (shown and not shown), the inventor realizes that in order to obtain preferred application of the wetness indicator in a relatively complicated pattern, the viscosity of the wetness indicator shall be chosen between 200 CPS and 2000 CPS at 90° C., the transfer roller 121 shall be kept at a temperature between 80° C. and 100° C. and the fluid holding tank 122 shall be kept at a temperature greater than 80° C. or the transfer roller 121 shall be kept at a temperature between 80° C. and 90° C. and the fluid holding tank 122 shall be kept at a temperature between 80° C. and 130° C. It was found by the inventor that those temperature ranges can ensure that no wrinkle will exist on the PE film after the wetness indicator is applied thereon.

FIG. 4 schematically illustrates a flow chart of a method according to an embodiment of the present application for forming a fabric sheet-like panel provided with a wetness indicator by the system. It should be understood by a person skilled in the art that some steps involved in the method can be coded as a program which can be stored in the memory, and can be revoked and executed by the central control unit 300 if necessary.

As shown, at Step S10, a wetness indicator is provided as a hot melt adhesive whose viscosity is chosen between 200 and 2000 CPS at 90° C. At Step S20, a system according to the embodiment of the present application is provided. At Step S30, a second functional film 2000 is enabled to pass through a transfer unit 100 of the system such that the wet indicator is applied onto the second functional film 2000 in a relatively complicated pattern not including any straight line. During application of the wetness indicator, a transfer roller 121 of the transfer unit 100 is maintained within a temperature range from 80° C. to 100° C. and a fluid holding tank 122 of the transfer unit 100 configured to transfer the wet indicator onto the second functional film via the transfer roller 121 is maintained within a temperate of greater than 80° C. or alternatively the transfer roller 121 is maintained within a temperature range from and the fluid holding tank 122 is maintained within a temperature range between 80° C. and 130° C. According to the present application, the relatively complicated pattern is configured such that the wetness indicator can be applied at a unit weight of 5 to 25 GSM by the transfer roller 121 and the fluid holding tank 122. At Step S40, a laminating unit 200 of the system is configured to receive the second functional film 2000 already applied with the wetness indicator and a first fabric substate 1000, which is supplied independently from the second functional film 2000; and laminate them together in such a way that the applied wetness indicator is therebetween, to form a fabric sheet-like panel adapted for producing diapers or pants-type diapers.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

The present application is not limited to the details of construction or the arrangements of components set forth in the foregoing description and/or the drawings. Various embodiments are provided solely for purposes of illustration, and the concepts described herein are capable of being practiced or carried out in other ways. Also, the phraseology and terminology used herein are for the purpose of description and should not be regarded as limiting. The use of “including,” “comprising,” “having,” “containing,” or “involving,” and variations thereof herein, is meant to encompass the items listed thereafter (or equivalents thereof) and/or as additional items.

1: A system for forming a fabric sheet-like panel having a wetness indicator, comprising:
a transfer unit (100) configured to receive a functional film (2000); and
a laminating unit (200) arranged downstream of the transfer unit (100) and configured to receive a fabric substate (1000) and laminate the same together with the functional film (2000) as the fabric sheet-like panel, wherein
the transfer unit (100) includes a rotatable transfer roller (121) and a fluid holding tank (122) fixedly disposed adjacent to the transfer roller (121), the fluid holding tank (122) is configured to store a wetness indicator which can be also as a hot melt adhesive, such that as the transfer roller (121) rotates, the wetness indicator is able to be applied in a patten including irregular curves onto a side of the functional film (2000) facing the fabric substrate (1000), and wherein
during application of the wetness indicator, the transfer roller (121) and/or the fluid holding tank (122) can be selectively heated such that a temperature of the transfer roller (121) is kept between 80° C. and 100° C. and a temperature of the fluid holding tank (122) is kept greater than 80° C.

2: The system as recited in claim 1, wherein the wetness indicator to be applied within the transfer unit (100) has a viscosity between 200 CPS and 2000 CPS at 90° C.

3: The system as recited in claim 1, wherein the transfer unit (100) is configured such that the wetness indicator is applied at a unit weight of 5 to 25 GSM onto the side of the functional film (2000).

4: The system as recited in claim 3, wherein the temperature of the transfer roller (121) is kept between 80° C. and 90° C. and the temperature of the fluid holding tank (122) is kept between 80° C. and 130° C.

5: The system as recited in claim 4, wherein the fabric substrate (1000) is a non-woven fabric substrate and the functional film (2000) is a PE film.

6: The system as recited in claim 5, wherein the transfer unit (100) further comprises a drive roller which is able to rotate in a direction opposite to the transfer roller (121), the PE film received by the transfer unit (100) is clamped and moved between the drive roller and the transfer roller (121), and the transfer roller (121) is on its outer surface formed with a pattern mirror relative to the pattern of the wetness indicator to be applied, and using the mirrored pattern, the wetness indicator is transferred onto the PE film.

7: The system as recited in claim 6, wherein the pattern of the wetness indicator is shaped as a figure which includes a sinuous line or an irregular line.

8: The system as recited in claim 7, wherein the mirrored pattern is embossed or recessed on the outer surface of the transfer roller (121).

9: The system as recited in claim 8, wherein the laminating unit (200) comprises a pressurizing roller (221) and an additional drive roller (211) which rotate in opposite directions such that both the non-woven fabric substrate and the PE film are received and laminated through between the two rollers.

10: A method for forming a fabric sheet-like panel having a wetness indicator, comprising:
providing a system including a transfer unit (100) configured to receive a functional film (2000) and a laminating unit (200) arranged downstream of the transfer unit (100) and configured to receive a fabric substate (1000) and laminate the same together with the functional film (2000);
providing a wetness indicator which has a viscosity between 200 CPS and 2000 CPS at 90° C. and storing it in a fluid holding tank (122) of the transfer unit (100);
moving the functional film (2000) through the transfer unit (100) such that the wetness indicator can be applied by a transfer roller (121) of the transfer unit (100) and the fluid holding tank (122), in a patten including irregular curves, onto a side of the functional film (2000) which will face the fabric substrate (1000) in the laminating unit (200); and
during application of the wetness indicator, keeping a temperature of the transfer roller (121) between 80° C. and 100° C. and keeping a temperature of the fluid holding tank (122) greater than 80° C.

11: The method as recited in claim 10, wherein in the transfer module (100) the wetness indicator is applied at a unit weight of 5 to 25 GSM onto the side of the functional film.

12: The method as recited in claim 11, wherein the temperature of the transfer roller (121) is kept between 80° C. and 90° C. and the temperature of the fluid holding tank (122) is kept between 80° C. and 130° C.

13: The method as recited in claim 12, wherein the fabric substrate (1000) is a non-woven fabric substrate and the functional film (2000) is a PE film.

14: The method as recited in claim 13, wherein the pattern of the wetness indicator is shaped as a figure which includes a sinuous line or an irregular line.
